(19) United States
(12) Patent Application Publication
(10) Pub. No.: US 2017/0369916 A1
(43) Pub. Date: Dec. 28, 2017

(54) MICROORGANISM HAVING ENHANCED CELLULOSE PRODUCTIVITY

(71) Applicant: Samsung Electronics Co., Ltd., Suwon-si (KR)

(72) Inventors: Jieun Kim, Suwon-si (KR); Hongsoon Rhee, Suwon-si (KR); Jiae Yun, Hwaseong-si (KR); Jinkyu Kang, Hwaseong-si (KR); Jinhwan Park, Suwon-si (KR)

(21) Appl. No.: 15/479,466

(22) Filed: Apr. 5, 2017

(30) Foreign Application Priority Data
Jun. 28, 2016 (KR) 10-2016-0081173

(51) Int. Cl. C12P 19/04; C12N 9/04; C07K 14/195

(52) U.S. Cl. CPC C12P 19/04; C12Y 101/05002; C07K 14/195; C12N 9/0006

(57) ABSTRACT

A genetically modified microorganism of the genus Gluconacetobacter has decreased pyrroloquinoline-quinone (PQQ)-dependent glucose dehydrogenase (GDH) activity of and increased glucose permease activity. The microorganism has enhanced productivity cellulose and is useful for the manufacture of microbial cellulose.

### CROSS-REFERENCE TO RELATED APPLICATION

This application claims the benefit of Korean Patent Application No. 10-2016-0081173, filed on Jun. 28, 2016, in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: 89,871 bytes ASCII (Text) file named “727555_ST25.TXT,” created Apr. 4, 2017.

### BACKGROUND

### 1. Field

The present disclosure relates to a microorganism having enhanced cellulose productivity, a method of producing cellulose using the same, and a method of producing the microorganism.

### 2. Description of the Related Art

Cellulose can be harvested from cultures of microorganisms. In the so-produced cellulose, glucose exists as a primary structure, β-1,4 glucan which forms a network structure of fibril bundles. This cellulose is also called ‘bio-cellulose or microbial cellulose’.

This microbial cellulose is typically pure cellulose, which is free of lignin or hemicellulose, unlike plant cellulose. The microbial cellulose, which is typically 100 nm or less in width, has a network structure of bundles of cellulose nanofibers, and has characteristic properties including, high water absorption and retention capacity, high tensile strength, high elasticity, and high heat resistance compared to plant cellulose. Due to these characteristics, microbial cellulose has been used in a variety of fields, including cosmetics, medical products, dietary fibers, audio speaker diaphragms, functional films.

Acetobacter, Agrobacteria, Rhizobia, or Sarcina microbes have been reported to produce cellulose. Of them, Komagataeibacter xylinum (also called ‘Gluconacetobacter xylinum’) is known as an excellent strain. Upon static culture of G. xylinum under aerobic conditions, cellulose with a three-dimensional network structure is formed as a thin film on the surface of the culture medium.

Despite the above-described developments, there is a demand for new genus Gluconacetobacter recombinant microorganisms having enhanced cellulose productivity and related methods.

### SUMMARY

An aspect of the disclosure provides a recombinant microorganism having enhanced cellulose productivity. In one embodiment, the microorganism is of the genus Gluconacetobacter and comprises a genetic modification that increases the activity of a glucose permease and a genetic modification that decreases activity of a pyrroloquinoline-quinone (PQQ)-dependent glucose dehydrogenase (GDH).

Another aspect provides a method of producing cellulose using the microorganism. The method includes culturing a recombinant microorganism of the genus Gluconacetobacter having enhanced cellulose productivity in a medium to produce cellulose. The microorganism comprises a genetic modification that increases activity of a glucose permease. Cellulose is collected from the culture.

Still another aspect provides a method of producing the microorganism comprising introducing an exogenous gene encoding glucose permease into a microorganism of the Gluconobacter genus.

### BRIEF DESCRIPTION OF THE DRAWINGS

These and/or other aspects will become apparent and more readily appreciated from the following description of the embodiments, taken in conjunction with the accompanying drawings in which:

FIG. 1 shows amounts of cellulose nanofiber (CNF) produced by strains of K. xylinus (Δgdh) expressing heterologous glucose permease genes; and

FIG. 2 shows amounts of cellulose nanofiber (CNF) produced by strains of K. xylinus (Δgdh) expressing homologous glucose permease genes.

### DETAILED DESCRIPTION

The term “parent cell” refers to a cell in a state immediately prior to a particular genetic modification, for example, a cell that serves as a starting material for producing a cell having a genetic modification that increases or decreases the activity of one or more proteins. A parent cell, thus, is a cell without a particular referenced genetic modification, but with the other genotypic and phenotypic traits of the genetically modified cell. Although the “parent cell” does not have the specific referenced genetic modification, the parent cell may be engineered in other respects and, thus, might not be a “wild-type” cell (though it may also be a wild-type cell if no other modifications are present). Thus, the parent cell may be a cell used as a starting material to produce a genetically engineered microorganism having an inactivated or decreased activity of a given protein (e.g., a protein having a sequence identity of about 95% or more to GDH) or a genetically engineered microorganism having an increased activity of a given protein (e.g., a protein having a sequence identity of about 95% or more to glucose permease). By way of further illustration, with respect to a cell in which a gene encoding GDH has been modified to reduce GDH activity, the parent cell may be a microorganism including an unaltered, “wild-type” GDH gene. The same comparison is applied to other genetic modifications. In performing a comparison to a genetically modified cell, a control cell having the genotype and phenotype of the parent cell may be used instead of an actual parental strain.

The term “increase in activity” or “increased activity”, as used herein, may refer to a detectable increase in an activity of a cell, a protein, or an enzyme. The “increase in activity” or “increased activity” may also refer to an activity level of a modified (e.g., genetically engineered) cell, protein, or enzyme that is higher than that of a comparative cell, protein, or enzyme of the same type, such as a cell, protein, or enzyme that does not have a given genetic modification (e.g., original or “wild-type” cell, protein, or enzyme). “Cell activity” may refer to an activity of a particular protein or enzyme of a cell. For example, an activity of a modified or engineered cell, protein, or enzyme may be increased by about 5% or more, about 10% or more, about 15% or more, about 20% or more, about 30% or more, about 50% or more, about 60% or more, about 70% or more, or about 100% or more than an activity of a non-engineered cell, protein, or enzyme of the same type, i.e., a wild-type cell, protein, or enzyme. An activity of a particular protein or enzyme in a cell may be increased by about 5% or more, about 10% or more, about 15% or more, about 20% or more, about 30% or more, about 50% or more, about 60% or more, about 70% or more, or about 100% or more than an activity of the same protein or enzyme in a parent cell (or control cell of equivalent genotype and phenotype to the parent cell). A cell having an increased activity of a protein or an enzyme may be identified by using any method known in the art.

An increase in activity of an enzyme or a polypeptide may be achieved by an increase in the expression or specific activity thereof. The increase in the expression may be achieved by introduction of a polynucleotide encoding the enzyme or polypeptide into a cell or by an increase in a copy number, or by a mutation (including point mutations and promoter-swaps) in the regulatory region of a homologous polynucleotide already present in the cell. The polynucleotide can express a gene. The microorganism to which the gene may be introduced can include the gene endogenously or may not include the gene. The gene may be operably linked to a regulatory sequence that allows expression thereof, for example, a promoter, an enhancer, a polyadenylation region, or a combination thereof. The polynucleotide whose copy number is increased may be endogenous or exogenous. The endogenous gene refers to a gene which has existed on a genetic material included in a microorganism. The exogenous gene refers to a gene that is introduced to a cell from the outside. The exogenous gene may be homologous or heterologous with respect to the host cell. The term “heterologous” means “foreign” or “not native.”

The “increase in the copy number” may be caused by introduction of an exogenous gene or amplification of an endogenous gene, and may be achieved by genetically engineering a cell so that the cell has a heterologous exogenous gene that does not exist in a non-engineered cell (e.g., wild-type, parental, or control cell lacking the). The introduction of the gene may be mediated by a vehicle such as a vector. The introduction may be a transient introduction in which the gene is not integrated into a genome, result in a stable episome, or may be an integration of the gene into the genome. The introduction may be performed, for example, by introducing a vector into the cell, the vector including a polynucleotide encoding a target polypeptide, and then replicating the vector in the cell, or by integrating the polynucleotide into the genome.

The introduction of the gene may be performed by a known method, such as transformation, transfection, and electroporation. The gene may be introduced via a vehicle or in itself. As used herein, the term “vehicle” refers to a nucleic acid molecule that is able to deliver other nucleic acids linked thereto. As a nucleic acid sequence mediating introduction of a specific gene, the vehicle used herein is construed to be interchangeable with a vector, a nucleic acid construct, and a cassette. Examples of the vector are a plasmid vector, a virus-derived vector, etc. A plasmid is a circular double-stranded DNA molecule linkable with another DNA. Examples of the vector may include a plasmid expression vector, and a virus expression vector, such as a replication-defective retrovirus, adenovirus, adeno-associated virus, or a combination thereof.

As used herein, the gene manipulation may be performed by molecular biological methods known in the art.

On the contrary, the term “inactivated” or “decreased” activity, as used herein, means that a cell has an activity of an enzyme or a polypeptide being lower than that measured in a parent, wild-type, or control cell (e.g., a non-genetically engineered cell). Also, the “inactivated” or “decreased” activity means that an isolated enzyme or a polypeptide has an activity being lower than that of an original or a wild-type enzyme or polypeptide. The inactivated or decreased activity encompasses no activity. For example, a modified (e.g., genetically engineered) cell or enzyme has enzymatic activity of converting a substrate to a product, which is decreased by about 5% or more, about 10% or more, about 20% or more, about 30% or more, about 40% or more, about 50% or more, about 55% or more, about 60% or more, about 70% or more, about 75% or more, about 80% or more, about 85% or more, about 90% or more, about 95% or more, about 98% or more, or about 100%, compared to that of a cell or enzyme that does not have the modification, i.e., a parent, wild-type, or control cell or enzyme. Decreased activity of an enzyme or a cell may be confirmed by any method known in the art. The inactivation or decrease includes the case that an enzyme has no activity or decreased activity even though the enzyme is expressed, or the case that an enzyme-encoding gene is not expressed or expressed at a low level, compared to a cell having a non-modified gene, i.e., a parent cell or a wild-type cell.

An activity of the enzyme may be inactivated or decreased by deletion or disruption of a gene encoding the enzyme. The “deletion” or “disruption” of the gene refers to mutation of part or all of the gene or part or all of a regulatory sequence of the gene, such as a promoter or a terminator region thereof, such that the gene is not expressed or is expressed at a reduced level, or expresses a gene product (e.g., enzyme) with no activity or reduced activity as compared to the naturally occurring gene product. The mutation may include addition, substitution, insertion, deletion, or conversion of one or more nucleotides of the gene. The deletion or disruption of a gene may be achieved by genetic manipulation such as homologous recombination, directed mutagenesis, or molecular evolution. When a cell includes a plurality of the same genes, or two or more different paralogs of a gene, one or more of the genes may be removed or disrupted. For example, inactivation or disruption of the enzyme may be caused by homologous recombination or may be performed by transforming the cell with a vector including a part of sequence of the gene, culturing the cell so that the sequence may homogonously recombine with an endogenous gene of the cell to delete or disrupt the gene, and then selecting cells, in which homologous recombination occurred, using a selection marker. CRISPR techniques can also be used as is known in the art.

The term “gene”, as used herein, refers to a nucleic acid fragment expressing a specific protein, and the fragment may or may not include a regulatory sequence of a 5′-non coding sequence and/or 3′-non coding sequence.

A “sequence identity” of a nucleic acid or a polypeptide, as used herein, refers to the extent of identity between bases or amino acid residues of sequences obtained after the sequences are aligned so as to best match in certain comparable regions. The sequence identity is a value that is obtained by comparison of two sequences in certain comparable regions via optimal alignment of the two sequences. A percentage of sequence identity may be calculated by, for example, comparing two optimally aligned sequences in the entire comparable regions, determining the number of locations in which the same amino acids or nucleic acids appear to obtain the number of matching locations, dividing the number of matching locations by the total number of locations in the comparable regions (e.g., the size of a range), and multiplying a result of the division by 100 to obtain the percentage of the sequence identity. The percentage of the sequence identity may be determined using a known sequence comparison program, for example, BLASTN (NCBI), BLASTP (NCBI), CLC Main Workbench (CLC bio), MegAlign™ (DNASTAR Inc), or EMBOSS Needle. For example, the Needleman-Wunsch global alignment algorithm in EMBOSS may be used with the default settings (gap opening penalty 10, gap extension penalty 0.5; end gap penalty=false, end gap open-10, for amino acid sequence comparisons, the BLOSUM62 matrix is used).

Various levels of sequence identity may be used to identify various types of polypeptides or polynucleotides having the same or similar functions or activities. For example, the sequence identity may include a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100%.

Where a polynucleotide sequence encodes a given protein, other polynucleotide sequences can be substituted due to the degeneracy of the genetic code.

The “genetic modification”, as used herein, includes an artificial alteration in a constitution or structure of a genetic material of a cell.

An aspect provides a genus Gluconacetobacter recombinant microorganism having enhanced cellulose productivity, the microorganism including a genetic modification of increasing activity of glucose permease.

Glucose permease is a membrane transport protein that facilitates glucose transport into or out of cells. The glucose permease may be exogenous or endogenous, and when exogenous, can be homologous or heterologous. The glucose permease may be in the form of a monomer consisting of a single polypeptide. The glucose permease may be selected from the group consisting of glucose permease (glcP) derived from the genus Bacillus, sodium/glucose cotransporter (sglT-3) derived from the genus Bacillus, glucose permease (glcP) derived from the genus Mycobacterium, glucose transporter (glf) derived from Zymomonas, sodium/glucose symporter (sglS) derived from the genus Vibrio, galactose permease (galP1) derived from the genus Gluconacetobacter, galactose permease (galP2) derived from the genus Gluconacetobacter, galactose permease (galP3) derived from the genus Gluconacetobacter, galactose permease (galP4) derived from the genus Gluconacetobacter, galactose permease (galP5) derived from the genus Gluconacetobacter, and glucose permease (gluP) derived from the genus Gluconacetobacter. The glucose permease may be selected from the group consisting of Bacillus pumilus glcP, Bacillus megaterium sglT-3, Bacillus licheniformis glcP, Mycobacterium smegmatis glcP, Zymomonas mobilis glf, Vibrio parahaemolyticus sglS, Gluconacetobacter xylinus galP1, Gluconacetobacter xylinus galP2₇ Gluconacetobacter xylinus galP3, Gluconacetobacter xylinus galP4, Gluconacetobacter xylinus galP5, and Gluconacetobacter xylinus gluP. The glucose permease may be a polypeptide having a sequence identity of about 95% or more (e.g., 96%, 97%, 98%, 99% or 100%) to an amino acid sequence of SEQ ID NO: 1, 2, 3, 4, 5, 6, 7 8, 9, 10, 11, or 12.

In the microorganism, the genetic modification may increase expression of a gene encoding glucose permease. The genetic modification may increase the copy number of the glucose permease gene. The genetic modification may increase the copy number of the gene encoding the polypeptide having a sequence identity of about 95% or more to the amino acid sequence of SEQ ID NO: 1, 2, 3, 4, 5, 6, 7, 8, 9, 10, 11, or 12. The gene may have a nucleotide sequence of SEQ ID NO: 13, 14, 15, 16, 17, 18, 19, 20, 21, 22, 23, or 24. The genetic modification may be introduction of the gene encoding glucose permease, for example, via a vehicle such as a vector. The gene encoding glucose permease may exist within or outside the chromosome. Also, multiple genes encoding a glucose permease may be introduced, which may be the same or different and may be in part of a single nucleic acid construct, or multiple separate constructs. For example, 2 or more, 5 or more, 10 or more, 50 or more, 100 or more, or 1000 or more genes (or copies of a gene) encoding glucose permease may be introduced.

The microorganism may be the genus Gluconacetobacter, for example, G. aggeris, G. asukensis, G. azotocaptans, G. diazotrophicus, G. entanii, G. europaeus, G. hansenii, G. intermedius, G. johannae, G. kakiaceti, G. kombuchae, G. liquefaciens, G. maltaceti, G. medeffinensis, G. nataicola, G. oboediens, G. rhaeticus, G. sacchari, G. saccharivorans, G. sucrofermentans, G. swingsii, G. takamatsuzukensis, G. tumulicola, G. tumulisoli, or G. xylinus (also called “Komagataeibacterxylinus”).

The microorganism can further include a genetic modification that decreases the activity of pyrroloquinoline-quinone:PQQ)-dependent glucose dehydrogenase (GDH). The microorganism may have deletion or disruption of a gene encoding GDH. The genetic modification can have deletion or disruption of a gene encoding a polypeptide having a sequence identity of about 95% or more to an amino acid sequence of SEQ ID NO: 25. The GDH gene may have a nucleotide sequence of SEQ ID NO: 26. Any of various known methods for deleting or disrupting genes may be used, some of which are illustrated in the Examples.

Another aspect provides a method of producing cellulose. The method includes culturing the recombinant microorganism of the genus Gluconacetobacter having enhanced cellulose productivity. The microorganism includes a genetic modification that increases the activity of glucose permease. The microorganism is cultured in a medium to produce cellulose; and the cellulose is collected from the culture.

The culturing may be performed in a medium containing a carbon source, for example, glucose. The medium used for culturing the microorganism may be any general medium that is suitable for host cell growth, such as a minimal or complex medium containing proper supplements. The suitable medium may be commercially available or prepared by a known preparation method.

The medium may be a medium that may satisfy the requirements of a particular microorganism depending on a product selected in the culturing. The medium may be a medium including components selected from the group consisting of a carbon source, a nitrogen source, a salt, trace elements, and combinations thereof.

The culturing conditions may be appropriately controlled for the production of a selected product, for example, cellulose. The culturing may be performed under aerobic conditions for cell proliferation. The culturing may be performed by static culture without shaking. The culturing may be performed at a low density. The density of the microorganism may be a density which provides intercellular space sufficient to not disturb secretion of cellulose by the cells of the culture.

The term “culture conditions”, as used herein, mean conditions for culturing the microorganism. Such culture conditions may include, for example, a carbon source, a nitrogen source, or an oxygen condition utilized by the microorganism. The carbon source that may be utilized by the microorganism may include monosaccharides, disaccharides, or polysaccharides. The carbon source may include glucose, fructose, mannose, or galactose as an assimilable sugar. The nitrogen source may be an organic nitrogen compound or an inorganic nitrogen compound. The nitrogen source may be exemplified by amino acids, amides, amines, nitrates, or ammonium salts. An oxygen condition for culturing the microorganism may be an aerobic condition of a normal oxygen partial pressure, a low-oxygen condition including about 0.1% to about 10% of oxygen in the atmosphere, or an anaerobic condition including no oxygen. A metabolic pathway may be modified in accordance with a carbon source or a nitrogen source that may be actually used by a microorganism.

The method may include collecting the cellulose from the culture. The separating may be, for example, collecting of a cellulose pellicle formed on the top of the medium. The cellulose pellicle may be collected by physically stripping off the cellulose pellicle or by removing the medium. The separating may be by collecting of the cellulose pellicle while maintaining its shape without damage.

Still another aspect provides a method of producing a microorganism having enhanced cellulose productivity, the method including introducing an exogenous gene encoding glucose permease into a genus Gluconacetobacter microorganism. The gene may be heterologous or endogenous. The introducing of the gene encoding glucose permease may comprise introducing a vehicle or vector including the gene into the microorganism.

The glucose permease encoded by the exogenous gene may be selected from the group consisting of glucose permease (glcP) derived from the genus Bacillus, sodium/glucose cotransporter (sglT-3) derived from the genus Bacillus, glucose permease (glcP) derived from the genus Mycobacterium, glucose transporter (glf) derived from Zymomonas, sodium/glucose symporter (sglS) derived from the genus Vibrio, galactose permease (galP1) derived from the genus Gluconacetobacter, galactose permease (galP2) derived from the genus Gluconacetobacter, galactose permease (galP3) derived from the genus Gluconacetobacter, galactose permease (galP4) derived from the genus Gluconacetobacter, galactose permease (galP5) derived from the genus Gluconacetobacter, and glucose permease (gluP) derived from the genus Gluconacetobacter. The glucose permease may be selected from the group consisting of Bacillus pumilus glcP, Bacillus megaterium sglT-3, Bacillus licheniformis glcP, Mycobacterium smegmatis glcP, Zymomonas mobilis glf, Vibrio parahaemolyticus sglS, Gluconacetobacter xylinus galP1, Gluconacetobacter xylinus galP2, Gluconacetobacter xylinus galP3, Gluconacetobacter xylinus galP4, Gluconacetobacter xylinus galP5, and Gluconacetobacter xylinus gluP. The glucose permease may be a polypeptide having a sequence identity of about 95% or more to an amino acid sequence of SEQ ID NO: 1, 2, 3, 4, 5, 6, 7, 8, 9, 10, 11, or 12.

The microorganism may include a genetic modification that decreases PQQ-dependent GDH, or the method may further include introducing a genetic modification of decreasing activity of pyrroloquinoline-quinone (PQQ)-dependent glucose dehydrogenase (GDH) into the microorganism. The genetic modification may be deletion or disruption of a gene encoding GDH.

The genus Gluconacetobacter recombinant microorganism having enhanced cellulose productivity be used to produce cellulose efficiently and with high yield.

In accordance with an embodiment, a method of producing the recombinant organism with enhanced cellulose efficiency comprises making the genetic modifications described above or in the Examples below.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Hereinafter, the present invention will be described in more detail with reference to Examples. However, these Examples are for illustrative purposes only, and the scope of the present invention is not intended to be limited by these Examples.

### Example 1.

### Preparation of Glucose Permease Gene-Including K. xylinus and Production of Cellulose

Komagataeibacter xylinus (Korean Culture Center of Microorganisms, KCCM 41431) and GDH gene-deleted K. xylinus were transformed with an exogenous (homologous or heterologous) glucose permease gene, and the transformants were cultured to produce cellulose. Observation of cellulose productivity for the various transformants was used to determine the effects of the various genes.

(1) Preparation of GDH Gene-Deleted K. xylinus

The membrane-bound pyrroloquinoline-quinone (PQQ)-dependent glucose dehydrogenase (GDH) gene in K. xylinus was inactivated by homologous recombination.

To delete the GDH gene by homologous recombination, fragments of the 5′- and 3′-ends of GDH gene were obtained by PCR amplification using a genomic sequence of K. xylinus as a template and a set of primers of GDH-5-F(SEQ ID NO: 27) and GHD-5-R(SEQ ID NO: 28) and a set of primers of GDH-3-F(SEQ ID NO: 29) and GHD-3-R(SEQ ID NO: 30). Further, a neo gene (nptll) fragment which is a kanamycin resistance gene derived from Tn5 was obtained by PCR amplification using a set of primers of SEQ ID NO: 32 and SEQ ID NO: 33. Three of the fragments of the 5′- and 3′-ends of GDH gene and the kanamycin resistance gene fragment were cloned into Sacl and Xbal restriction sites of a pGEM-3zf vector (#P2271, Promega Corp.) using an In-fusion HD cloning kit (#PT5162-1, Clontech) to prepare pGz-dGDH. This vector thus obtained was transformed into K. xylinus by electroporation. The transformed K. xylinus strain was spread on a HS-agar medium (0.5% peptone, 0.5% yeast extract, 0.27% Na₂HPO₄, 0.15% citric acid, 2% glucose, and 1.5% bacto-agar) supplemented with 100 pg/ml of kanamycin, and then cultured at 30° C. Selection on kanamycin yielded a GDH deletion strain, designated K. xylinus (Δgdh).

(2) Introduction of Glucose Permease Gene

Bacillus pumilus glcP, Bacillus megaterium sglT-3, Bacillus licheniformis glcP, Mycobacterium smegmatis glcP, Zymomonas mobilis glf, Vibrio parahaemolyticus sglS, Gluconacetobacter xylinus galP1, Gluconacetobacter xylinus galP2, Gluconacetobacter xylinus galP3, Gluconacetobacter xylinus galP4, Gluconacetobacter xylinus galP5, or Gluconacetobacter xylinus gluP genes, namely, a nucleotide sequence of SEQ ID NO: 13, 14, 15, 16, 17, 18, 19, 20, 21, 22, 23, or 24 were individually introduced into K. xylinus and K. xylinus (Δgdh), respectively. Bacillus pumilus glcP, Bacillus licheniformis glcP, Mycobacterium smegmatis glcP, or Zymomonas mobilis glf is a glucose-proton symporter, Gluconacetobacter xylinus galP1, Gluconacetobacter xylinus galP2, Gluconacetobacter xylinus galP3, Gluconacetobacter xylinus galP4, and Gluconacetobacter xylinus galP5 are galactose-proton sym porters, Gluconacetobacter xylinus gluP is a glucose/galactose transporter, Bacillus megaterium sglT-3 or Vibrio parahaemolyticus sglS is a sodium/glucose symporter.

Glucose permease genes derived from the respective microorganisms were obtained by 12 PCR reactions using 12 primer sets (SEQ ID NOS: 34 and 35; SEQ ID NOS: 36 and 37; SEQ ID NOS: 38 and 39; SEQ ID NOS: 40 and 41; SEQ ID NOS: 42 and 43; SEQ ID NOS: 44 and 45; SEQ ID NOS: 46 and 47; SEQ ID NOS: 48 and 49; SEQ ID NOS: 50 and 51; SEQ ID NOS: 52 and 53; SEQ ID NOS: 54 and 55; SEQ ID NOS: 56 and 57); using genomic DNA of Bacillus pumilus glcP, Bacillus megaterium sglT-3, Bacillus licheniformis glcP, Mycobacterium smegmatis glcP, Zymomonas mobilis glf, Vibrio parahaemolyticus sglS, and K. xylinus as a template.

Each gene obtained by PCR was cloned into the Pstl restriction site of pCSa (SEQ ID NO: 31) using an In-fusion HD cloning kit (#PT5162-1, Clontech) to allow expression under Tac promoter. Each vector thus obtained was transformed into K. xylinus by electroporation. The transformed K. xylinus strain was spread on an HS-agar medium (0.5% peptone, 0.5% yeast extract, 0.27% Na₂HPO₄, 0.15% citric acid, 2% glucose, and 1.5% bacto-agar) supplemented with 100 μg/ml of chloramphenicol, and then cultured at 30° C. Selection on chloramphenicol yielded glucose permease-overexpressing strains.

(3) Test of Glucose Consumption and Cellulose Production

Control and transformed K. xylinus strains were inoculated into a 250-mL flask containing 50 ml of HS medium (0.5% peptone, 0.5% yeast extract, 0.27% Na₂HPO₄, 0.15% citric acid, and 2-4% glucose), respectively and cultured at 230 rpm at 30° C. for 5 days. Then, glucose consumption and the product cellulose were quantified. During culturing of the glucose permease gene-overexpressing recombinant strains, 100 μg/ml of chloramphenicol was added to media. Glucose was analyzed by high performance liquid chromatography (HPLC) equipped with an Aminex HPX-87H column (Bio-Rad, USA), and cellulose production was measured after washing the cellulose solid formed in the flask with 0.1 N sodium hydroxide and water and then drying the cellulose solid in an oven at 60° C.

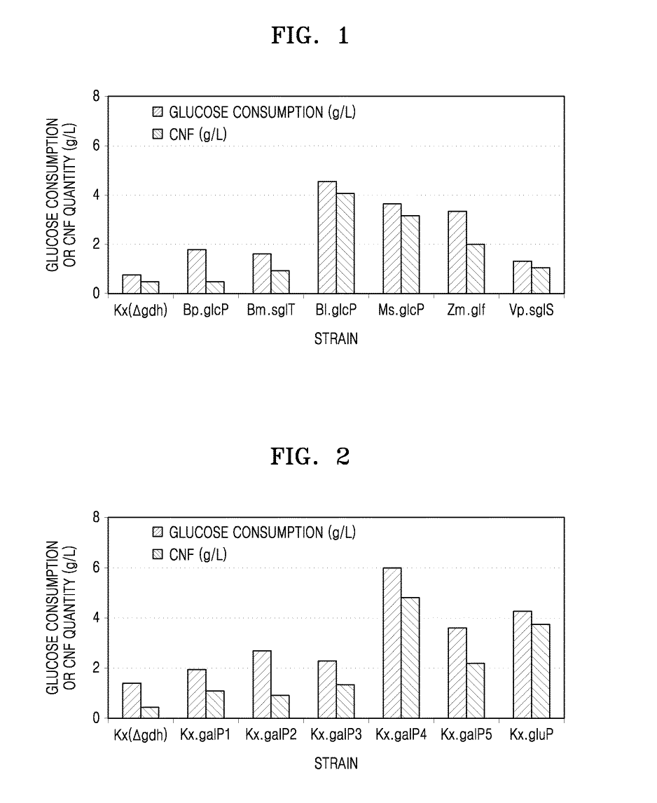

The results are given in FIGS. 1 and 2, which show amounts of cellulose nanofiber (CNF) produced by each glucose permease gene-expressing K. xylinus(Δgdh) strain under shaking culture, and glucose consumption. As shown in FIG. 1, when each of the foreign (i.e., heterologous) glucose permease genes was introduced into K. xylinus(Δgdh), CNF production amount showed about a 1.1 to about 6.9-fold increase, and glucose consumption showed about 1.8 to about a 6.8-fold increase, indicating that the introduced foreign glucose permeases affects transport of glucose into cells and cellulose production in the strain.

As shown in FIG. 2, when each of the homologous glucose permease genes was introduced into K. xylinus(Δgdh), CNF production amount showed about a 2.1 to about 12.1-fold increase, and glucose consumption showed about a 1.4 to about 4.3-fold increase, indicating that the introduction of homologous glucose permeases affect transport of glucose into cells and cellulose production in the strain. In FIGS. 1 and 2, Kx(Δgdh), and Bp.glcP, Bm.sglt, Bl.glcP, Ms.glcP, Zm.glf, Vp.SglS, Kx.galP1, Kx.galP2, Kx.galP3, Kx.galP4, Kx.galP5, and Kx.gluP represent K. xylinus(Δgdh), and strains prepared by introducing K. xylinus(Δgdh) with Bacillus pumilus glcP, Bacillus megaterium sglT-3, Bacillus licheniformis glcP, Mycobacterium smegmatis glcP, Zymomonas mobilis glf, Vibrio parahaemolyticus sglS, Gluconacetobacter xylinus galP1, Gluconacetobacter xylinus galP2, Gluconacetobacter xylinus galP3, Gluconacetobacter xylinus galP4, Gluconacetobacter xylinus galP5, and Gluconacetobacter xylinus gluP, respectively. Tables 1 and 2 represent the results of FIGS. 1 and 2, respectively.

TABLE 1

| | Strain | | | | | | |
|---|---|---|---|---|---|---|---|
| Item | Kx(Δgdh) | Bp.glcP | Bm.sglt | Bl.glcP | Ms.glcP | Zm.glf | Vp.SglS |
| Glucose | 0.72 | 1.80 | 1.62 | 4.51 | 3.66 | 3.32 | 1.33 |
| consumption (g/L) | | | | | | | |
| CNF(g/L) | 0.46 | 0.50 | 0.90 | 4.07 | 3.15 | 2.01 | 1.06 |

TABLE 2

| | Strain | | | | | | |
|---|---|---|---|---|---|---|---|
| Item | Kx(Δgdh) | Kx.galP1 | Kx.galP2 | Kx.galP3 | Kx.galP4 | Kx.galP5 | Kx.gluP |
| Glucose | 1.40 | 1.92 | 2.62 | 2.27 | 5.99 | 3.58 | 4.24 |
| consumption (g/L) | | | | | | | |
| CNF(g/L) | 0.40 | 1.09 | 0.85 | 1.31 | 4.84 | 2.16 | 3.76 |

It should be understood that embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variation thereof is encompassed by the invention unless otherwise indicated herein or otherwise clearly contradicted by context.

What is claimed is:

1. A recombinant Gluconacetobacter microorganism, the microorganism comprising a genetic modification that increases glucose permease activity, and a genetic modification that decreases pyrroloquinoline-quinone (PQQ)-dependent glucose dehydrogenase (GDH) activity compared to a parent cell and having enhanced cellulose productivity.

2. The microorganism of claim 1, wherein the genetic modification is an increase in the copy number of a gene encoding the glucose permease by introduction of an exogenous polynucleotide encoding the glucose permease, increasing the copy number of a polynucleotide encoding an endogenous glucose permease gene, or by a mutation in the regulatory region of an endogenous glucose permease gene.

3. The microorganism of claim 1, wherein the glucose permease is selected from the group consisting of glucose permease (glcP) from the genus Bacillus, sodium/glucose cotransporter (sglT-3) from the genus Bacillus, glucose permease(glcP) from the genus Bacillus, glucose permease (glcP) from the genus Mycobacterium, glucose transporter (glf) from Zymomonas, sodium/glucose symporter (sglS) from the genus Vibrio, galactose permease (galP1) from the genus Gluconacetobacter, galactose permease (galP2) from the genus Gluconacetobacter, galactose permease (galP3) from the genus Gluconacetobacter, galactose permease (galP4) from the genus Gluconacetobacter, galactose permease (galP5) from the genus Gluconacetobacter, and glucose permease (gluP) from the genus Gluconacetobacter.

4. The microorganism of claim 2, wherein the gene encoding the glucose permease is selected from the group consisting of genes of glucose permease (glcP) from the genus Bacillus, sodium/glucose cotransporter (sglT-3) from the genus Bacillus, glucose permease(glcP) from the genus Bacillus, glucose permease (glcP) from the genus Mycobacterium, glucose transporter (glf) derived from Zymomonas, sodium/glucose symporter (sglS) from the genus Vibrio, galactose permease (galP1) from the genus Gluconacetobacter, galactose permease (galP2) from the genus Gluconacetobacter, galactose permease (galP3) from the genus Gluconacetobacter, galactose permease (galP4) from the genus Gluconacetobacter, galactose permease (galP5) from the genus Gluconacetobacter, and glucose permease (gluP) from the genus Gluconacetobacter.

5. The microorganism of claim 1, wherein the glucose permease is a polypeptide having a sequence identity of about 95% or more to an amino acid sequence of SEQ ID NO: 1, 2, 3, 4, 5, 6, 7, 8, 9, 10, 11, or 12.

6. The microorganism of claim 2, wherein the gene encoding the glucose permease has a nucleotide sequence of SEQ ID NO: 13, 14, 15, 16, 17, 18, 19, 20, 21, 22, 23, or 24.

7. The microorganism of claim 1, wherein the genetic modification increases expression of a gene encoding the glucose permease.

8. The microorganism of claim 1, wherein the microorganism is Gluconacetobacter xylinus.

9. The microorganism of claim 1, wherein the genetic modification that decreases GDH activity is deletion or disruption of a gene encoding GDH.

10. The microorganism of claim 9, wherein the GDH is a polypeptide having a sequence identity of 95% or more to an amino acid sequence of SEQ ID NO: 25.

11. The microorganism of claim 9, wherein the gene encoding GDH has a nucleotide sequence of SEQ ID NO: 26.

12. A method of producing cellulose, the method comprising:
culturing the recombinant microorganism of claim 1, in a medium to produce cellulose; and
collecting the cellulose from a culture.

13. The method of claim 12, wherein the glucose permease is selected from the group consisting of glucose permease (glcP) from the genus Bacillus, sodium/glucose cotransporter (sglT-3) from the genus Bacillus, glucose permease(glcP) from the genus Bacillus, glucose permease (glcP) from the genus Mycobacterium, glucose transporter (glf) from Zymomonas, sodium/glucose symporter (sglS) from the genus Vibrio, galactose permease (galP1) from the genus Gluconacetobacter, galactose permease (galP2) from the genus Gluconacetobacter, galactose permease (galP3) from the genus Gluconacetobacter, galactose permease (galP4) from the genus Gluconacetobacter, galactose permease (galP5) from the genus Gluconacetobacter, and glucose permease (gluP) from the genus Gluconacetobacter.

14. The method of claim 12, wherein the microorganism is G. xylinus.

15. The method of claim 12, wherein the glucose permease is a polypeptide having a sequence identity of 95% or more to an amino acid sequence of SEQ ID NO: 1, 2, 3, 4, 5, 6, 7, 8, 9, 10, 11, or 12.

16. The method of claim 12, wherein the genetic modification is an increase in the copy number of a gene encoding a polypeptide having a sequence identity of 95% or more to an amino acid sequence of SEQ ID NO: 1, 2, 3, 4, 5, 6, 7, 8, 9, 10, 11, or 12.

17. The method of claim 12, wherein the microorganism comprises a genetic modification that decreases activity of PQQ-dependent GDH.

18. A method of producing a microorganism having enhanced cellulose productivity, the method comprising introducing an exogenous gene encoding glucose permease into microorganism of the Gluconacetobacter genus.

19. The method of claim 18, wherein the microorganism comprises a genetic modification that decreases PQQ-dependent GDH, or the method further comprises introducing to the microorganism a genetic modification that decreases PQQ-dependent GDH.


SEQUENCE LISTING (57 sequences)

SEQ ID NO: 1 - protein, 395 aa, Bacillus pumilus
MKKVFYFGCVFYFFIGTIHVFFGSLTPYLLASYDKGPGELSSLIFFQFIGFLTGVLLSPILVRKKGYGAVLTMGLLLMIGSLLLGLLVPGWSTLVLAGFFLGSGAGSLETTAGAYVISMANSAKRISIMEVFFGLGALLFPLVILLTVNEQTWHYVFLFQVGALTFFLMLWIAFMNKLPRGQMISPSNGVKKPSLLVDRNNRIIVVIMICFAFFYAGIETNFANFLPSIMLEKGGDNWGLFAVSTFWTAIVIGRTVIARKADHLHPLRFLKLSAALMILLLVIFALTTHITAQLLLIFFIGLCAAGMFPIALTASALMIENAIDEATSYFIAAASLGGACLSFLIGFSLEWAGAASAIFVFAFLAVLLFAAAIQMNRVKKKETVLPKQSALKADQ

SEQ ID NO: 2 - protein, 570 aa, Bacillus megaterium
MQNAKKPFRSDTQQARPEKSNETSDFSGKSNRNNRLLTPLWTTIIGFFIFMVVAFIYSLYNPDLYWPGLILMFIMYGVIYFIGARAAASKKGKSDDMLVAGRSMPLWISMFTMTATWVGGGYIAGTAETVYSSGLTWTQAPWCYSISLILGGIFFARKMRRFEFMTMLDPLESRFGKKMATVLYFPAILGELFWSAAILTALGTTFGVILGLSFSISIILSALIAIAYTVIGGLWAVAHTDILQLSIMFLGLFLVLPFAFSNTGGVGAVFSTYSEGMTGSLHLFPPLKGWEDPKWGNTYWQWWDSTFLLIFGGIPWQIYFQRVLSAKNEKAAMWLSITAGIFCALAALPPTLIGMIGYSADWSSFGASSPESASMILTYVFKYLTPDLVGAIALGGLAAAVMAAVAASLLSASGMAAWNVYRPIVKPNATQAQLDKVIKRSIIIIGTGATLIALNSESVYSLWYLSGDLVYCILFPQLVCALFFKGANWYGSLAGFIVSLVLRIGGGEPLLHLKALLPYPMIEDGVVMFPFRTFAMVGGLLTIFIVSYATRRICPPQPLRNLHRDISVEK

SEQ ID NO: 3 - protein, 394 aa, Bacillus licheniformis
MKKIFLFGCSFYFLVGVIHILLGSLSPYIIQEYQRDLHDLSFLIFFQFTGFLNGVLLAPMFVRRTSHTAVLTFGLLLILVTLLGVFLFDMFIFFVIMGFLLGFGAGTLETTMGAYVIAQDKNAKGMNILEVFFGLGALLFPFLIYILTERYAWHFPLYALFIFVFVLACMWVVYLRRKTPGTASGQMAYQEKPTVSAIFETGRKEKNIFLFLIFAFVYAGIETNFANFLPALMLEKGAEEISVISVTFFWTGMVCGRLLTSIFGGRITSVAFLIFSAGALTVLLLILAWFPVHQTQLLLVFFIGLSAAGIFPCAVTLASLAGKPFTEEITSLFISSASLGGALLSFLIGWAIDASAAAVFPFLLFGGLGGLLLAISAVIFLSGLQKNKQSHLDM

SEQ ID NO: 4 - protein, 498 aa, Mycobacterium smegmatis
MNVIGITLLPRGRIMSHGPVSDDTPSIFGDDDQAASSGRTAVRIAAVAALGGLLFGYDSAVINGAVDSIQEDFGIGNYALGLAVASALLGAAAGALSAGRIADRIGRIAVMKIAAVLFFISAFGTGFAPETVTLVVFRIVGGIGVGVASVIAPAYIAETSPPGIRGRLGSLQQLAIVLGIFTSFVVNWLLQWAAGGPNEVLAMGLDAWRWMFLAMAVPAVLYGALAFTIPESPRYLVATHKIPEARRVLSMLLGQKNLEITITRIRDTLEREDKPSWRDLKKPTGGIYGIVWVGLGLSIFQQFVGINVIFYYSNVLWQAVGFSADQSAIYTVITSVVNVLTTLIAIALIDKIGRKPLLLIGSSGMAVTLATMAVIFANATVKPDGTPDLPGASGLIALIAANLFVVAFGMSWGPVVWVLLGEMFPNRFRAAALGLAAAGQWAANWLITVSFPELRNHLGLAYGFYALCAVLSFLFVSKWVEETRGKNLEDMHAEALGH

SEQ ID NO: 5 - protein, 473 aa, Zymomonas mobilis
MSSESSQGLVTRLALIAAIGGLLFGYDSAVIAAIGTPVDIHFIAPRHLSATAAASLSGMVVVAVLVGCVTGSLLSGWIGIRFGRRGGLLMSSICFVAAGFGAALTEKLFGTGGSALQIFCFFRFLAGLGIGVVSTLTPTYIAEIRPPDKRGQMVSGQQMAIVTGALTGYIFTWLLAHFGSIDWVNASGWCWSPASEGLIGIAFLLLLLTAPDTPHWLVMKGRHSEASKILARLEPQADPNLTIQKIKAGFDKAMDKSSAGLFAFGITVVFAGVSVAAFQQLVGINAVLYYAPQMFQNLGFGADTALLQTISIGVVNFIFTMIASRVVDRFGRKPLLIWGALGMAAMMAVLGCCFWFKVGGVLPLASVLLYIAVFGMSWGPVCWVVLSEMFPSSIKGAAMPIAVTGQWLANILVNFLFKVADGSPALNQTFNHGFSYLVFAALSILGGLIVARFVPETKGRSLDEIEEMWRSQK

SEQ ID NO: 6 - protein, 543 aa, Vibrio parahaemolyticus
MSNIEHGLSFIDIMVFAIYVAIIIGVGLWVSRDKKGTQKSTEDYFLAGKSLPWWAVGASLIAANISAEQFIGMSGSGYSIGLAIASYEWMSAITLIIVGKYFLPIFIEKGIYTIPEFVEKRFNKKLKTILAVFWISLYIFVNLTSVLYLGGLALETILGIPLMYSILGLALFALVYSIYGGLSAVVWTDVIQVFFLVLGGFMTTYMAVSFIGGTDGWFAGVSKMVDAAPGHFEMILDQSNPQYMNLPGIAVLIGGLWVANLYYWGFNQYIIQRTLAAKSVSEAQKGIVFAAFLKLIVPFLVVLPGIAAYVITSDPQLMASLGDIAATNLPSAANADKAYPWLTQFLPVGVKGVVFAALAAAIVSSLASMLNSTATIFTMDIYKEYISPDSGDHKLVNVGRTAAVVALIIACLIAPMLGGIGQAFQYIQEYTGLVSPGILAVFLLGLFWKKTTSKGAIIGVVASIPFALFLKFMPLSMPFMDQMLYTLLFTMVVIAFTSLSTSINDDDPKGISVTSSMFVTDRSFNIAAYGIMIVLAVLYTLFW

SEQ ID NO: 7 - protein, 474 aa, Komagataeibacter xylinum
VNDDTVKHDDLSYRDSVQQGRRNAFLFAGAAGLAGLMFGLDTGVIAGALKFMGLDLGANERAQEWIVSSLMLGAAGGSLLAIPVSHYRGRRGAMFYAGLLFLLGTALCSLAPSIPVMIAGRVCLGIGVGFASFSAPLYIAEITEKSQRGTMISLYQLVITAGMLLALLSDSLLSYGGHWRWMLGILAVPTMFFILATTRVPYSPRWLAMHGRRREARGVLQKVRGSRERANNELDRIEQNLRKTKGNGFQLLKTSRGFRKTLALGMALQMFQQLAGINILLYYAPHLLEHLGFSAQAAVWCTTLLGLANMVATGVAIVLIDRWGRRPLLLLSTLMASSSLCAFGFVLFAHVEGSMGSIAIIGLLVLFTLGYALGEGPVPWTMCTEIQPLQGRGLAIACSTFANWMTNWLISNVFLSVMSLIGDYGIFWLLAGFNAVFFLIGYFLVPETRGCSLEEIEQRVNAGYPLREIGQPGG

SEQ ID NO: 8 - protein, 472 aa, Komagataeibacter xylinum
VSQPVSSPIATPCPPPASPPGATGVRAALTTAMAGLLVGLDTGLIAEALGFIGHDFHASARAQEWVVSVLMMGALLGSLGAGVFSRRFGRRLALGTATVLIGAGALLCATAGLIGQILLGRFLIGVAIGICTFTAPLYISELTTGKMRGTMVSTFSMLQSCGILLGYLAGGLLAGGGHWRLMVGLPVVPALALFAACAVLPSSPSWLAARGRFEEARKVLRDLRGDEAEADRELDCIRHELGAGKAVSGFALLRAKPYFRRSVALGIGLQIMQQLTGINVVMYYAPKILEGAHFGTAAAAWATVLVGLVNAVVSMGAIYLVSRWGRRPLLVSSCVIMACALGCAAMIEGMHLQGLGATLSLMAALLVFVAGFGMGAGPLVWTLCSEIQPIAGRDFGVACSTLANWGMDWAVSNTFLTIVAAMGAGWTFAGFSLMNIGFVLFTVLLVPETRDVPLEVIEQNLEAGLPLRRIGR

SEQ ID NO: 9 - protein, 493 aa, Komagataeibacter xylinum
MPEDDLVSRAMTHASPQGQATSPATPTTGHAIVVGVLAALAGLMFGLDTGVIAGALRFIGTDFDASPRMQEWIVSSMMAAAAVGSLIAGTISFRFGRRRALLGSSILFLLGSLISALAPSVTVLIIGRIFLGFAVGIAAFTAPLYISEVSAVAQRGSMIACYQLMMTGGIFLSYVTDGVLANGAHWRWMLGLMTVPATVFLIGCLFLPDSPRWLMMRGEKLRARTVMRYLRPSPQQADQEISDIATELTRGRSEGFSFFRNNANFRRSVGLGIVLQIMQQLTGINVLMYYAPKVFQAADFGASAAGWATALIGLINLVATCVAIVTVDRWGRRPLLLLSCAIMTGSMLLAGGLVEYGGHDTTAQIAMVGSLLVFVLGFAIGAGPLVWTLCAEIQPLRGRDFGIVCSTFTNWATNWAVSNTFLSVLDTLGEAHTFWLFAGMNALFIAITLFYVPETKGVSLENIESHLLAGWPLRDLGARSMPQDAKISTRPSA

SEQ ID NO: 10 - protein, 471 aa, Komagataeibacter xylinum
MENQPAPPVFDSARMRTLIIGCLAALAGLMAGLDIGVISGALDLLAATFHATTFQQEWIVSAMMGGAAAGSLCGGWMSHQIGRKHALLVGAAVFVAGSLACALAWSIPSMIAGRLIMGFAIGVAAFTAPLYLSEIASEQARGAMISTYQLMITAGIFIAFLSNTMFSYTGNWRGMFAIAAVPGVLFLIGVLFLPYSPRWLMMRGRRKEALEVLEDLRNDKSVAMQEIQNISRQLQQKQRGWSLLRNNSNFRRSIFLGMTLQVMQQLAGVNVVMYYAPKIFSLAGYVGPAQMWCTAMVGLVNMLATFIAIGLVDRWGRKPILYTGFLIMAVGMGSLGFMLNRPHLDQTEQIIAVFMLLIYISGFAMSAGPLMWVLCSEVQPLQGRDLGISISTLTNWIANMIVGASFLSLLQWMGNGPTFWLFAGFNLFFVLVTWRFIPETRDMSLEKIEQRLMAGLPLREIGQGIPLPQEK

SEQ ID NO: 11 - protein, 479 aa, Komagataeibacter xylinum
HRQPGIAQTGQPAGSHHPPAIRGRAGLIGGLAALSGILFGLDTGVMSGALDLIAQEFTLSDLQRESIVAIMLLGAALGVMAAAWLSHTWGRKRTLVLTAGLFVIGPLLCAEASSFGTLLFARLLLGVATGATTFTTPLYIAEIADSGRRGTMILGYQLMISCGLLAAYVSDGLFSYFGVWRWMLGIVGFPGLVFMMGVMFLPPSPRWLLAQGRERDARRVLIELRGLPRLVMAERNAIMARLAARKDGIGNFMHDPNCRRAMWLAVGLQVAQQFSGINAVLYYAPYIIGLVGYSHYVQVWGPVGVGVINLLSTFVATFWVDRIGRRPMLIGGFAVMALAMAGQAMILAGGVPPMPGLRLVMGVCMLVFVAAFAFSAGPLAWLLCAEILPLRGREFGMACSTCANWIANMVVSATFLTGLEVLGAGWVLWVYAALNVVFMAMVALRVPETRGMTLEQIEAELMRGTKLRALGRNAPPEQH

SEQ ID NO: 12 - protein, 449 aa, Komagataeibacter xylinum
MGGGVLPLRGDDGNGQRGECMEKANSGQDGGGGPGGAYGTRPLLVMAGLFFIIGFVTWLNGPLITFVQVAFGVGPVGAFLVPMCFYLAYFFCAFPAMALARRTGLRGGIRLALGVMAAGTLGFGECVGRGWYAGALAGLSVLGAGLTLLQVAVNPYVTLLGPAAQAARRIAGMGIANKLSGIIAPIIFSLLVMHDIGGVVARLAASGNARMHAQVLAGFAHAVVLPYRGMAVVLLLVALGLRHAGLPDLRLACRDAAPPGGRMAGMAWVGIAVVFVYVGVEVMAGDGIGLYARGMGLLVGQTRFLTAFTLAGMLGGYVLGSFMVPAVIRSAPYLGLSALVGGALCTGAIMAHGMGSVLCIALLGVANAMMMPILFPLVLHMAGAWRQRANALLVMAFCGGAVMPQCFALLQGPWGMKPAFMGLVMPGYGVIGLFALVVWWRARGLGRAA

SEQ ID NO: 13 - DNA, 1188 bp, Bacillus pumilus
atgaaaaaagtattttattttggctgtgtcttttatttttttattgggaccattcatgtgttttttggcagcttaactccttatttactggctagttacgataagggtcccggggaattatcttctttaatcttttttcagtttattggttttttgacaggggttctgttatcccccatccttgtgaggaaaaaaggctatggcgctgttctgactatggggcttttgctgatgattggatcacttctgcttgggcttttggtgccgggctggtcaactcttgtgctggcaggtttttttcttggcagtggagcaggcagtcttgagacaacagcaggagcgtatgtgatttcgatggcaaacagtgcgaagcgaatcagcatcatggaggtcttctttgggttaggagcgctattatttccacttgtgattttactgactgtcaacgaacagacgtggcactatgtgtttttatttcaagtcggtgcactaactttcttccttatgctttggattgcttttatgaacaaattgcctcgtggacagatgatttctccttccaatggggtgaaaaaaccgtccttacttgttgatcgaaacaatcgaatcattgtggtgatcatgatttgctttgcctttttctacgcagggattgaaacgaactttgcgaactttttgccgtcgatcatgctggagaagggaggagacaattggggtctctttgcagtctccactttctggacagccattgtcatcggcagaaccgtgattgcgagaaaagcagatcatttgcacccgctgcgttttttaaagctaagtgcagcactcatgattctgctgctcgtgatctttgcgctgacaacacacatcaccgctcagctgcttctcatctttttcatcggcctgtgtgcagctggtatgttcccaatcgcactgactgcatctgcattaatgattgaaaatgccatcgacgaggccacgagttactttattgcagccgcaagtttaggcggagcctgcttgtccttcttgatcggctttagtcttgaatgggcaggagcagcaagtgccatctttgttttcgccttcttagctgttcttctatttgcagctgcgattcaaatgaatcgtgtgaagaaaaaagaaaccgttctccccaagcagtcggcactgaaagcggatcagtaa

SEQ ID NO: 14 - DNA, 1713 bp, Bacillus megaterium
atgcaaaatgctaaaaagccatttcgttcagacacacaacaagccaggcctgaaaagtcgaatgagacttctgacttttcagggaaatcaaacagaaacaaccgtctactaacccccttatggacaactattataggatttttcatctttatggttgtcgcatttatctattctctctataatccagatctctattggccaggcctcattttaatgttcattatgtatggtgttatttattttattggtgctcgagctgctgcaagtaaaaaaggaaaatcagatgatatgcttgttgcgggaagatctatgccgctatggatttcaatgttcacaatgaccgctacttgggtaggtggagggtatattgctggaacggccgaaactgtttattcctcaggactgacttggacgcaagcgccatggtgttattcaatcagcttaattttaggcggtatcttttttgctagaaagatgagaaggtttgagtttatgacaatgctcgatcctctagaatctcgtttcggtaagaaaatggctacggttctttattttccagctatattaggagagctgttttggagcgcagctatcttaactgctttaggaacaacattcggtgtgattttaggcctcagtttttcaatttccatcattctttccgcacttattgccattgcatacaccgtgattggcggcttgtgggcagtagcacataccgatatcttacagctttctattatgtttttaggattatttctggtgcttccttttgcattttcgaatacgggaggggtgggagccgttttttctacttattcagaaggtatgactggttctcttcatttatttcctccattaaaaggctgggaagatccaaaatggggaaatacatattggcaatggtgggatagcacatttttacttatttttggtggtattccatggcagatttattttcagcgcgtgttatctgctaaaaatgaaaaagcagcaatgtggctatctattacagccggcattttttgcgctctagcggccttgcctccaactttaataggaatgattggttacagtgcggactggtcttcatttggagcgtcaagccctgagagcgcgtctatgattttaacatacgtatttaaatatctaacgcctgatttagtaggagcgattgcgcttggaggactagctgctgccgtcatggctgcagtagcggcttcgttactttcagcttcgggaatggcagcttggaacgtgtatcgtccgattgtaaaaccaaatgcgacacaggcgcagttagataaagttattaaacgttctatcattatcattggaactggagcaactttaattgctttaaactctgagagtgtctactccttatggtatttatcaggggacctagtgtattgtattctttttccacaattggtttgcgctttgttctttaaaggagcaaactggtacggatctttggctggattcattgtgtctcttgttcttcgaatcggtggaggggaaccgctgcttcatttaaaagcgctgcttccgtatccgatgatagaagacggagtggtcatgtttccatttcgtacatttgcgatggtaggcgggttgttaactatttttattgtttcttacgcaacgcgacgtatttgccctcctcagccgcttcgaaaccttcatcgtgacatatcggttgagaaataa

SEQ ID NO: 15 - DNA, 1185 bp, Bacillus licheniformis
atgaaaaaaatatttctattcggctgttcgttttattttctagtaggggttatccatattctgctggggagcctgtcaccgtacatcattcaagaatatcaacgggatcttcatgatctatccttcctgatctttttccaattcaccggttttctaaacggcgtcctgcttgcgccgatgtttgtcagacgcacctctcatacggctgtattgacgtttggccttcttcttattctcgttacgcttttaggtgttttcctttttgatatgttcatcttttttgtcatcatgggatttttgctcggctttggcgcgggtactttagaaacgacgatgggggcgtatgtgattgcgcaagataaaaatgcgaaagggatgaatattttagaggtttttttcggattaggcgcgctgctgtttccttttcttatttatatccttacggaacgatatgcctggcatttccccttgtatgctttatttatcttcgtttttgtgctcgcgtgtatgtgggtggtttatttgcgcagaaaaacccccggcactgcttccggtcagatggcttatcaggaaaaaccgactgtatcagccatatttgaaaccggaaggaaagaaaaaaacattttcctctttctcatattcgcttttgtgtatgcgggtatcgagaccaatttcgcaaactttttgccggcgctgatgctggaaaaaggggctgaagaaatcagcgtgatcagcgtcacgtttttttggacggggatggtatgcggacgtttattgacaagtatttttggcggacgcataacttccgttgcctttctgatcttcagcgccggagccttgactgttttgcttttgattctcgcgtggtttccggttcatcaaacacagctgctcctcgtatttttcatcgggctgtcggcagccggcatttttccgtgcgccgtcactcttgcctcgttggctggaaagccttttacagaggaaatcacgagtctcttcatttcgtccgcaagtctgggaggagcgcttctttcattcttgatcggctgggcgattgatgcaagcgcagccgctgtcttcccgtttttgctgttcggcggattggggggcttgctgctggcgatcagcgcggtgatttttttatccggcctgcaaaaaaacaagcagagtcatttggatatgtag

SEQ ID NO: 16 - DNA, 1497 bp, Mycobacterium smegmatis
atgaacgtgatcggcatcaccctgctgccgcgcgggcgcatcatgagccacgggccggtgagcgacgacaccccgtcgatcttcggggacgatgatcaggcggcctcctccgggcgcaccgccgtccgcattgcggcggtcgcggccctgggcgggctgctgttcggctacgacagcgccgtcattaatggggccgtggactcgatccaggaggacttcggcatcggcaattacgccctggggctggcggtggcgtcggcgctgctgggggcggccgccggcgcgctgtcggccggccggatcgccgaccgcatcgggcgcatcgcggtgatgaaaatcgccgccgtcctgttcttcatcagcgccttcggcacggggttcgcccccgaaacggtcaccctggtggtgttccgcatcgtcgggggcatcggcgtgggcgtggcctcggtgatcgcccccgcctacattgccgagaccagcccgccgggcatccggggccgcctgggctcgctgcagcagctggccatcgtgctgggcatcttcacgtcctttgtcgtcaactggctgctgcagtgggcggcgggcggccccaacgaggtgctggcgatgggcctggacgcgtggcgctggatgttcctggccatggccgtgccggccgtcctgtatggggcgctggcgttcaccatcccggagtcgccgcggtatctggtggccacgcacaagatcccggaagcgcgccgggtgctgagcatgctgctggggcagaagaacctggagatcaccatcacgcggatccgggacaccctggagcgcgaggataaaccgtcgtggcgcgatctgaagaagcccaccggcgggatctacgggatcgtgtgggtcggcctgggcctgtcgatcttccagcagttcgtcggcatcaatgtgatcttctactattcgaatgtgctgtggcaggccgtcggcttcagcgccgatcagtccgcgatctataccgtgattacgtcggtggtcaacgtgctgacgacgctgattgcgatcgcgctgatcgacaagattggccgcaagccgctgctgctgattggcagctccggcatggcggtcacgctggccaccatggcggtcattttcgccaatgccacggtcaagcccgatggcacgcccgatctgcccggcgcgtccggcctgattgcgctgattgcggcgaacctgttcgtggtcgcgttcggcatgtcctgggggccggtggtgtgggtgctgctgggggaaatgttccccaaccgctttcgggcggccgcgctgggcctggcggcggccgggcagtgggccgcgaactggctgattaccgtgagctttcccgaactgcgcaaccatctgggcctggcctatggcttttatgccctgtgcgcggtgctgtcctttctgtttgtgagcaagtgggtggaagaaacccggggcaagaacctggaagatatgcatgccgaagcgctggggcattga

SEQ ID NO: 17 - DNA, 1422 bp, Zymomonas mobilis
atgagttctgaaagtagtcagggtctagtcacgcgactagccctaatcgctgctataggcggcttgcttttcggttacgattcagcggttatcgctgcaatcggtacaccggttgatatccattttattgcccctcgtcacctgtctgctacggctgcggcttccctttctgggatggtcgttgttgctgttttggtcggttgtgttaccggttctttgctgtctggctggattggtattcgcttcggtcgtcgcggcggattgttgatgagttccatttgtttcgtcgccgccggttttggtgctgcgttaaccgaaaaattatttggaaccggtggttcggctttacaaattttttgctttttccggtttcttgccggtttaggtatcggtgtcgtttcaaccttgaccccaacctatattgctgaaattcgtccgccagacaaacgtggtcagatggtttctggtcagcagatggccattgtgacgggtgctttaaccggttatatctttacctggttactggctcatttcggttctatcgattgggttaatgccagtggttggtgctggtctccggcttcagaaggcctgatcggtattgccttcttattgctgctgttaaccgcaccggatacgccgcattggttggtgatgaagggacgtcattccgaggctagcaaaatccttgctcgtctggaaccgcaagccgatcctaatctgacgattcaaaagattaaagctggctttgataaagccatggacaaaagcagcgcaggtttgtttgcttttggtatcaccgttgtttttgccggtgtatccgttgctgccttccagcagttagtcggtattaacgccgtgctgtattatgcaccgcagatgttccagaatttaggttttggagctgatacggcattattgcagaccatctctatcggtgttgtgaacttcatcttcaccatgattgcttcccgtgttgttgaccgcttcggccgtaaacctctgcttatttggggtgctctcggtatggctgcaatgatggctgttttaggctgctgtttctggttcaaagtcggtggtgttttgcctttggcttctgtgcttctttatattgcagtctttggtatgtcatggggccctgtctgctgggttgttctgtcagaaatgttcccgagttccatcaagggcgcagctatgcctatcgctgttaccggacaatggttagctaatatcttggttaacttcctgtttaaggttgccgatggttctccagcattgaatcagactttcaaccacggtttctcctatctcgttttcgcagcattaagtatcttaggtggcttgattgttgctcgcttcgtgccggaaaccaaaggtcggagcctggatgaaatcgaggagatgtggcgctcccagaagtag

SEQ ID NO: 18 - DNA, 1632 bp, Vibrio parahaemolyticus
atgtcgaacatcgagcacggcctgagcttcatcgatatcatggtcttcgccatctacgtcgccatcattattggcgtcgggctgtgggtgagccgggataagaagggcacccagaagtccacggaggactacttcctggccggcaagagcctgccgtggtgggcggtcggggcgtcgctgatcgcggccaatattagcgcggaacagtttattggcatgagcgggtccggctattccattggcctggcgattgcgagctatgaatggatgtcggccatcacgctgatcatcgtggggaagtactttctgccgatcttcatcgagaagggcatctacaccatcccggagttcgtggagaagcgcttcaacaagaagctgaagacgatcctggccgtgttctggatctccctgtacatcttcgtgaacctgacctccgtcctgtacctgggggggctggccctggagaccatcctggggatcccgctgatgtactccatcctggggctggcgctgttcgcgctggtgtactcgatctacggggggctgtcggccgtcgtctggaccgacgtcatccaggtgttcttcctggtgctgggggggtttatgaccacctacatggccgtgagcttcattgggggcacggacggctggttcgcgggggtgagcaaaatggtcgatgccgcgccgggccattttgaaatgatcctggaccagtccaacccccagtacatgaacctgccgggcattgccgtcctgattggcggcctgtgggtcgccaatctgtattattggggcttcaaccagtatatcatccagcggacgctggcggccaagtcggtctcggaagcgcagaagggcatcgtgttcgccgcgtttctgaagctgatcgtgccgttcctggtggtcctgcccggcattgccgcgtatgtgattacctcggacccccagctgatggccagcctgggggatattgcggccacgaatctgccctccgcggcgaatgcggataaagcctatccgtggctgacccagtttctgccggtgggcgtgaaaggcgtggtgtttgcggcgctggcggcggccattgtgagctcgctggcctcgatgctgaattcgacggccaccatttttaccatggatatttataaagaatatatcagcccggactcgggcgaccataagctggtgaacgtggggcgcaccgccgcggtggtggccctgattatcgcgtgcctgatcgcccccatgctgggcggcatcggccaggcctttcagtatatccaggaatatacgggcctggtgagcccgggcatcctggcggtcttcctgctgggcctgttctggaaaaagacgacctccaagggggcgatcatcggcgtcgtcgcctcgatccccttcgccctgttcctgaagttcatgcccctgtccatgcccttcatggatcagatgctgtatacgctgctgttcacgatggtggtgatcgccttcacgtccctgagcacgtcgatcaacgatgacgaccccaagggcatctccgtgacgtcgtcgatgttcgtcaccgaccgcagcttcaacatcgcggcgtatggcatcatgatcgtgctggcggtgctgtataccctgttctggtga

SEQ ID NO: 19 - DNA, 1425 bp, Komagataeibacter xylinum
gtgaatgacgatactgtaaagcatgatgacctgtcctatcgggacagcgtgcaacaaggacgccggaacgcctttctttttgccggggcggctggacttgccgggcttatgtttggtctcgacaccggtgtcattgccggtgcgctcaaattcatgggccttgacctcggggccaatgagcgcgcgcaggaatggattgtctcctcgctcatgctgggtgccgcaggcggctcgctgctggccatcccggtttcgcattaccgggggcggcggggggccatgttctatgccggcctgctgttcctgcttggcaccgccctgtgttcgctggccccgtccattcccgtcatgatcgcaggccgtgtctgccttggcattggcgttggtttcgcgtctttttcagcaccgctctacattgctgaaattaccgaaaagagccagcgcggcaccatgatctcgctctaccagcttgtcattaccgcaggcatgctcctggccctgctgtcagacagcctgctttcttatggcgggcactggcggtggatgctgggtattctggccgtgcccacgatgttcttcattctggccacgacgcgggtgccttattcaccacgctggctggccatgcatgggcgcaggcgcgaagcccgtggcgtgttgcaaaaagtgcgtggctcacgcgaacgcgccaataatgaacttgaccggattgaacagaacctccgcaaaaccaagggaaatggcttccaactcctcaaaacctcaaggggttttcgcaagacactggccctgggcatggcgctccagatgttccagcaactcgcgggcatcaatatcctgctgtattacgccccgcatctgcttgaacatcttggtttttcggctcaggcggcagtatggtgcacgacactgctcggcctcgccaacatggtggcgaccggcgtagctattgtgctgatcgaccgctgggggcgcaggccgttgctgctgctcagcacgctcatggcctcttccagcctgtgtgcgttcgggttcgtgctgtttgcgcatgtggagggcagcatgggcagcattgccatcattgggctgctggtgctgttcacgctgggctatgccctgggggaagggccggtgccgtggaccatgtgcaccgagatccagcccctgcaggggcgcgggctggccatagcgtgctccaccttcgccaactggatgaccaactggctcatcagcaacgttttcctgtccgtcatgagtcttatcggggattacggcattttctggcttctggctggtttcaatgcggttttcttcctgatcggttacttccttgtgcctgaaacgcgcggctgctcgcttgaagagatcgagcagcgtgtcaacgcgggctaccccttgcgtgaaatcgggcaaccgggaggctaa

SEQ ID NO: 20 - DNA, 1419 bp, Komagataeibacter xylinum
gtgtcacagcccgtatcttcgcccatcgccacgccatgccccccaccggcttcgcctccgggcgccacgggggtgcgcgcggcgctgaccaccgccatggcgggcctgctcgtagggcttgataccgggctgatcgccgaggcgctgggctttatcggccacgattttcacgccagcgcgcgcgcgcaggaatgggtcgtgtcggtgctgatgatgggcgcgctgctcggctcgctcggggcgggggtgttctcacgccgctttggccgcaggctggcgctgggcacggcgaccgtgctgatcggggcaggcgcgctgctttgcgccacggccgggctgatcgggcagatcctgctggggcggttcctgatcggggtggcgattggcatctgcacctttaccgcgccgctctacatatcggaactgaccacaggcaagatgcgtggcaccatggtctccaccttttccatgctccagtcatgcggcatcctgctgggctatctggcgggtggcctgcttgcgggcggcgggcactggcggctgatggtggggctaccggtggtgccagccctggcgctgttcgccgcgtgcgcggtgctgccttccagcccgtcatggcttgcggcgcgcgggcgctttgaggaggcgcgcaaggtgctgcgcgacctgcggggcgatgaagccgaggccgaccgcgaacttgactgcatccgccacgaactcggcgcgggcaaggccgtaagcggctttgccctgttgcgggccaagccgtatttccgccgctcggtggcgctgggcatagggttgcagatcatgcagcaactcaccggcattaacgtcgtgatgtattacgcccccaagattctggagggcgcgcattttggcaccgccgccgccgcatgggccacggtgctggtcgggctggtcaatgcggtggtgagcatgggagccatctatctggtctcgcgctgggggcgcaggccgctgctggtctcaagctgcgtcatcatggcctgcgccctgggttgcgctgccatgatcgaggggatgcacctgcaggggctgggcgccacgttgagcctgatggcggcgcttctggtttttgtggcgggctttggcatgggggcggggccgctggtgtggaccctgtgctccgaaatccagcccatagcagggcgtgactttggcgtggcgtgctcaaccctggccaactggggcatggactgggcggtgagcaatacctttctcaccattgtcgcggcaatgggggcggggtggacctttgccgggttcagcctgatgaatatcggtttcgtgctgtttaccgtgctgctggtgcccgagacgcgcgacgtgccgctggaggtgatcgagcagaacctcgaggctggcctgcccctgcgccgcattggccgctag

SEQ ID NO: 21 - DNA, 1482 bp, Komagataeibacter xylinum
atgcccgaagacgatctggtttcccgcgccatgacacatgccagtccacaggggcaggccacctctcctgccacgccaacgacgggacatgccattgtggtgggggtgctggcggcccttgccggcctcatgttcgggctggataccggagtgattgcgggcgcgctgcgctttattggcacggattttgatgcatcgccacgcatgcaggaatggattgtctcctccatgatggcggcggcggccgtggggtcactgattgccgggacaatctcgttccgttttggccgcaggcgtgcgctgctggggtcgtccattctttttctgcttggctccctgatcagcgcattggccccatccgtcacggtgctcatcatcggtcggattttcctgggcttcgcggtggggatcgcggcctttacggcgccgctctacatatccgaagtttcggcggtggcgcagcgcgggtcgatgatcgcgtgttaccagttgatgatgacgggcggcatcttcctgtcctacgtgacggatggggtgctggcgaatggcgcgcactggcggtggatgctgggactcatgacagtgcccgcgaccgtcttcctcatcggctgcctgttcctgccggacagcccgcgctggctcatgatgcgcggggaaaaactgcgtgcccgcacggtgatgcgctacctgcggccaagcccgcagcaggcggaccaggaaatatccgatattgccacggaactgacccgaggccggtcggaagggttttcgtttttccgcaacaacgccaacttccgccgctcggtcgggttgggcatcgtgttgcagatcatgcagcagctgaccggcatcaatgtgctgatgtattacgcgcccaaagtctttcaggccgccgatttcggcgcatccgccgcaggctgggccacggcgctgatcggtctgatcaacctcgtcgccacatgcgtcgcaatcgtgaccgttgaccgctggggccgccggccgctgctgctgctcagctgcgccatcatgacaggcagcatgctgctggcaggtggcctggttgaatatggtggccatgacaccacagcccagatcgccatggtcgggtcactgctggtgtttgtgctggggttcgccattggggccgggccactggtctggacattgtgcgctgaaatccagcccctccgcggtcgtgactttggcattgtctgctccacctttaccaactgggcgaccaactgggcggtcagcaatacattcctcagcgtgctcgacacattgggcgaagcccataccttctggctgtttgctggcatgaatgccctgtttatcgccatcacgctgttttatgtgccggaaacaaaaggggtttcgctggaaaatattgaatcgcacctgcttgccggctggcccctgcgcgaccttggcgcgcgctccatgccgcaggacgcaaaaatatccacacgtccatctgcctga

SEQ ID NO: 22 - DNA, 1416 bp, Komagataeibacter xylinum
atggaaaatcagcccgcgccccccgtatttgattcggcacggatgcgtaccctcatcattggttgccttgctgcgctggcgggcctcatggccgggctggatatcggggtcatatccggtgcgcttgacctgcttgccgccacctttcacgcaacgaccttccagcaggaatggattgtcagcgccatgatgggtggtgctgccgcaggctcgctgtgcgggggctggatgtcgcaccagatcgggcgcaagcacgcgctgctggtgggggcggccgtgttcgtggcaggctcgcttgcctgtgcgctggcatggtcgatcccgtccatgatcgcagggcggctcatcatggggtttgccatcggggtggccgcgttcacggcgccgctctacctgtccgagattgcaagcgagcaggcgcgtggcgccatgatctcgacctaccagctcatgattacggcgggcattttcatcgccttcctcagcaacaccatgttcagctatacgggtaactggcgcggcatgttcgccattgccgccgtgccgggcgtgctgttcctgattggcgtgctgttcctgccctacagcccgcgctggctcatgatgcgtggccgccgcaaggaagcgctggaagtgctggaagacctgcgcaacgacaaaagcgtggccatgcaggagatccagaacatcagccgccagttgcagcagaagcagcgcggctggagcctgctgcgcaacaacagcaacttccgccgctccatctttctgggcatgacgctgcaggtcatgcagcagctcgcgggcgtgaacgtggtgatgtactacgcccccaagatcttctcgcttgcaggctatgtcggccccgcgcagatgtggtgcacggccatggtggggctggtgaacatgctggccacctttattgccatcggccttgtcgatcgctgggggcgcaagccgatcctgtacacgggcttcctgatcatggccgtgggcatgggcagccttggcttcatgctcaaccgcccgcatctggaccagacggagcagatcatcgcggtgttcatgctgctgatctatatttccggcttcgccatgtcggcgggtccgctgatgtgggtgctgtgctcggaggtgcagccgctgcaggggcgtgaccttggcatttccatctccacgctcaccaactggattgccaacatgatcgtgggcgcgagcttcctgtcgctgttgcagtggatgggcaatggccccaccttctggctgtttgcggggttcaacctgttcttcgtgctggttacatggcgcttcattcccgagacacgggacatgtcgcttgaaaagatcgagcagcgcctgatggcgggcctgccgctgcgcgaaatcgggcagggcataccgctgccgcaggagaaataa

SEQ ID NO: 23 - DNA, 1443 bp, Komagataeibacter xylinum
atgcacaggcagcccggtatcgcgcagacaggccagcccgcaggctcccaccaccccccggccatacgtggccgcgcagggctgatcggggggctggcggccctttcaggcatcctgttcgggctggatacgggggtcatgtccggcgcgctcgacctgatcgcgcaggaattcaccctgtcggacctgcagcgcgaatcgatcgtggccatcatgctgctcggtgccgcccttggcgtgatggccgccgcatggctctcgcacacatgggggcgcaagcgcacgctggtgctcacggcggggctgttcgtgatcggcccgctgctctgtgccgaggcttcatcattcggcacgctcctgttcgcccgcctgcttcttggcgtggccacgggggccaccacgttcaccaccccgctctatattgccgagattgccgattccggccgccggggcacgatgatcctgggctatcagctcatgatctcgtgcgggctgctcgcggcctatgtgtcggatgggctgttttcctattttggcgtgtggcggtggatgctgggcatcgtggggtttccgggccttgtgttcatgatgggggtcatgttcctgccgcccagcccgcgctggctgctggcccaggggcgcgagcgtgacgcgcggcgcgtgctgatcgaactgcgcggcctgccccggctggtcatggccgagcgcaacgccatcatggcccggctggcggcacgcaaggacggtatcggcaatttcatgcatgaccccaactgccgccgcgccatgtggctggcggtgggcctgcaggtggcgcagcagttctcgggcatcaacgcggtgctgtactacgcgccctacatcatcgggctggtgggctacagccattacgtgcaggtgtgggggccggtgggggtgggggtgatcaacctgctttcaacctttgtggcgacgttctgggtggaccggatcgggcgcaggcccatgctgattggcggattcgcggtcatggcgctggccatggcggggcaggccatgatcctggcaggcggcgtgccgcccatgccggggctgcggctggttatgggggtgtgcatgctagtgtttgtcgcggccttcgcgttctcggccgggccgctggcgtggctgctctgcgcggagatcctgccgctgcgcgggcgcgagttcggcatggcgtgctccacctgcgccaactggatcgccaacatggtggtcagcgccacgttccttaccgggcttgaggtgctgggggcagggtgggtgctgtgggtctatgcggcgctcaacgtggtgttcatggccatggtggcgctgcgcgtgcccgagacgcggggcatgacgctggagcagatcgaggcggaactcatgcgcggcacgaagctgcgcgcgctgggccgcaatgcgccgcccgaacagcactag

SEQ ID NO: 24 - DNA, 1350 bp, Komagataeibacter xylinum
atgggcggtggtgtcctgcctttgcgtggcgacgatgggaatgggcagcggggggaatgcatggaaaaagcgaattcaggccaggacggagggggcgggccaggcggggcttatggcacgcggccactgttggtcatggccgggctgtttttcatcatcgggtttgtcacatggctcaacgggccactgatcacgttcgtgcaggttgcctttggcgtggggccggttggggcgtttctggtgccgatgtgtttttacctcgcttattttttctgcgcgtttcctgccatggcgcttgcgcggcgcacggggctgcgtggtggcatcaggctggcgcttggtgtcatggcggcaggcacgcttgggtttggcgaatgtgtggggcgcggctggtatgctggtgcgcttgcgggcctgtcggtgctgggcgcggggctgacgctgctgcaggtggcggtcaatccgtatgtgacgctgcttggccctgcggcacaggcggcgcggcgcattgcagggatgggcattgccaataagctttcaggaattatcgcacctataatattttctcttctggtcatgcatgatattggcggcgtggtggcccgtctcgcggccagcggcaatgcccgcatgcacgcacaggttctggccggtttcgcccatgcggtggtgctgccctaccggggcatggcggttgtgctgctgcttgtggcgctggggctgcggcatgcgggactgcccgatctgcgtctggcctgccgggatgcagccccgcccggcgggcgcatggcgggcatggcctgggtcgggattgccgtggtgttcgtgtatgtaggggtggaggtgatggcgggcgatggcattggcctatatgcgcgcggcatggggcttctggtcgggcagacgcggtttctcaccgcgttcacgcttgcgggcatgctgggcgggtatgtgctgggcagtttcatggtgcctgccgtgatccggtccgcgccctatctgggcctgtccgcgcttgtgggaggcgcgttgtgcaccggggccatcatggcccacggcatgggctcggtgctgtgcatcgccctactgggagtggccaatgccatgatgatgccaatcctgtttccgctggtgctgcacatggcgggggcgtggcggcagcgggcgaatgcgctgctggtcatggcgttttgcggcggcgctgtcatgccgcagtgttttgccctgctgcaggggccgtggggcatgaaaccggccttcatggggctggtcatgcccggttatggggtgatcggactttttgcgctggtggtatggtggcgcgcgcgggggttgggtcgtgccgcgtga

SEQ ID NO: 25 - protein, 796 aa, Gluconacetobacter xylinus
MNSLMRSAPLLAAAIAVCALTGLYLLGGGLWLCLIGGSFYYVVAGVLLLVTAVLLARRQAMALTVYAVLLLGTMVWAVQEAGFDFWALAPRGDILVPIGIVLALPWVTRHLQPASPATHLPLFGAIGAAVVVVGAALTQDPQDIAGSLPPVAQNAPEPGDAHQMPDEDWQAYGRTQFGDRFSPLKQVNASNVGKLKVAWTFRTGDLRGPNDPGEITDEVTPIKIRDTLYLCTPHQILFALDAKTGQQRWKFDPKLAYNPTFQHLTCRGVSYHEDRADDAQAADGAAAPAECARRIFLPTNDGQLFALDAATGARCASFGNNGVVNLQDGMPVKTLGFYEPTSPPVVTDTTVIVSGAVTDNYSTHEPSGVTRGFDVHTGALKWAFDPGNPDPNEMPSEHHTFVPNSPNSWITSSYDAKLDLIYIPMGVQTPDIWGGNRGADAERYASSIVALNATTGRLVWSYQTVHHDLWDMDIPAQPSLVDIRNEQGEVIPTLYAPAKTGNIFVLDRRNGQPVVPAPEHPVPQGAAPGDHVSPTQPFSELSFRPKKLLTDADMWGGTMYDQLVCRIMFHRLRYEGTFTPPSLQGTLVFPGNLGMFEWGGLAVDPVRQIAIANPIAIPFVSKLIPRGPNNPATPDKSLPSGSESGVQPQFGVPYGVDLHPFLSPFGLPCKQPAWGYMSGIDLRTNKIVWKHRNGTIRDSAPLPLPIKMGVPSLGGPLTTAGGVAFLTSTLDYYIRAYDVTNGQVLWQDRLPAGGQSTPMTYAVDGKQYIVTADGGHGSFGTKLGDYIVAYSLPDGN

SEQ ID NO: 26 - DNA, 2391 bp, Gluconacetobacter xylinus
atgaatagcctcatgcgctcggctccccttctcgctgcggccattgccgtctgcgccctgacgggtctctacctgctgggaggcgggctatggctgtgtctcatcggcggctccttttattatgttgtcgccggtgtgctgctgctggtcacggccgtgctgctggcgcggcggcaggccatggcgcttacggtctatgccgtgctcctgctcggcacgatggtgtgggccgtgcaggaagccgggtttgatttctgggcgctcgcaccgcggggcgatattctggtgcccatcggcatcgtgctcgccctgccgtgggtcacacgtcacctgcagcctgccagccccgccacccacctgcccctgttcggcgcaattggcgccgccgtggtcgtcgttggcgcggccctgacgcaggacccgcaggatatcgcgggcagcctgcccccagtcgcgcagaatgcccccgagccgggcgatgcccaccagatgcctgatgaggactggcaggcctatggccgcacccagttcggtgaccggttctccccgctcaagcaggtcaatgccagtaatgtcggcaaactgaaggtggcctggaccttccgcaccggcgacctgcgcggccccaatgaccccggtgaaatcaccgatgaggtcacccccatcaagatccgtgatacgctctatctgtgcaccccccaccagatcctgttcgcgctcgatgcgaagaccggccagcagcggtggaagtttgaccccaagctggcctacaaccccaccttccagcacctgacctgccgtggcgtgtcctatcatgaggacagggcggatgacgcgcaggcagccgatggtgccgcagccccggccgagtgcgcgcgccgcatcttcctgcccaccaatgatggccagcttttcgcgctcgatgccgcaaccggcgcgcgctgcgcaagctttggcaataatggcgtggtgaacctgcaggacggcatgccggtcaagacgctgggcttttatgaaccgacctcccccccggtcgtgaccgataccaccgtgatcgtgtccggcgccgtgaccgacaactattccacgcatgagccttcgggggttacgcgcggcttcgacgtgcataccggcgcgctgaaatgggcgttcgaccccggcaatcccgatccgaacgagatgccgtccgagcaccacaccttcgtgccgaactcacccaattcgtggatcacgtcgtcctatgatgccaagctggacctgatctacatccccatgggcgtgcagacgcccgatatctggggcggcaaccgcggcgccgatgccgagcgctatgcaagctccatcgtggcgctgaacgccaccaccggcaggctggtctggtcctaccagaccgtgcaccacgacctgtgggacatggacatccccgcccagcccagcctggtcgatatccgcaacgaacagggcgaggtcatccccaccctgtatgccccggccaagaccggcaacatcttcgtgcttgaccggcgcaacggccagcccgtggtgcccgcccccgagcacccggtgccgcagggcgcagcccctggcgatcacgtttcgcccacgcagcctttctcggagctgagcttccgccccaagaagctgctgaccgatgccgatatgtggggcggcacgatgtatgaccagctggtctgccgcatcatgttccaccgcctgcgctacgaaggcacattcacgccgccttcgctgcagggcacgctggtcttccccggcaatctcggcatgttcgaatggggcggccttgcggtcgaccccgtgcgccagatcgcgattgccaaccccatcgccattccgttcgtctccaaactgatcccgcgcggcccgaacaacccggcaacgcctgacaagtccctgccctcgggctcggagagtggcgtgcagccgcagtttggcgtgccttacggcgtggacctgcatccgttcctctcgccgtttggcctgccgtgcaagcagcccgcctggggctacatgtcgggcatcgacctgcgcaccaacaagatcgtgtggaagcaccgcaacggcacgatccgtgacagcgcaccgctgcccctgcccatcaagatgggcgtgcccagccttggcggcccgctcaccacggcgggtggcgtggccttcctcacttccacgctcgattactacatccgcgcctatgacgtgacgaacggccaggtgctgtggcaggaccgcctgcctgccggtggccagtccacgcccatgacctatgcggtcgatggcaagcagtacatcgtcacggccgatggcggccacgggtcgttcggcaccaaactcggcgactacatcgtcgcctacagcctgcctgacgggaactga

SEQ ID NO: 27 - DNA, 35 bp, Artificial Sequence
  Synthetic gdh 5 prime terminal forward primer
tagaatactcaagcttggagctaccagaccgtcca

SEQ ID NO: 28 - DNA, 33 bp, Artificial Sequence
  Synthetic gdh 5 prime terminal reverse primer
tcagaccccgtagaacaaacatgccaaggttgc

SEQ ID NO: 29 - DNA, 33 bp, Artificial Sequence
  Synthetic gdh 3 prime terminal forward primer
caacaccttcttcacttgaatggggtggccttg

SEQ ID NO: 30 - DNA, 35 bp, Artificial Sequence
  Synthetic gdh 3 prime terminal reverse primer
tatagggcgaattcgggcaggcggtcctgccacag

SEQ ID NO: 31 - DNA, 3128 bp, Artificial Sequence
  Synthetic pCSa vector
gaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaagtcggacgcgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaaaggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacatagatctcccgggtaccgagctctctagaaagaaggagggacgagctattgatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttttttaaggcagttattggtgcccttaaacgcctggttgctacgcctgaataagtgataataagcggatgaatggcagaaattc

SEQ ID NO: 32 - DNA, 34 bp, Artificial Sequence
  Synthetic Km-F primer
tcacgccgccttcgcgtgaagaaggtgttgctga

SEQ ID NO: 33 - DNA, 33 bp, Artificial Sequence
  Synthetic Km-R primer
aacaccagcgtgcccttctacggggtctgacgc

SEQ ID NO: 34 - DNA, 36 bp, Artificial Sequence
  Synthetic BP.glcP-F
tagagtcgacctgcaatgaaaaaagtattttatttt

SEQ ID NO: 35 - DNA, 36 bp, Artificial Sequence
  Synthetic BP.glcP-R
ccaagcttgcatgccttactgatccgctttcagtgc

SEQ ID NO: 36 - DNA, 36 bp, Artificial Sequence
  Synthetic BM.sglt-F
tagagtcgacctgcaatgcaaaatgctaaaaagcca

SEQ ID NO: 37 - DNA, 36 bp, Artificial Sequence
  Synthetic BM.sglt-R
ccaagcttgcatgccttatttctcaaccgatatgtc

SEQ ID NO: 38 - DNA, 36 bp, Artificial Sequence
  Synthetic Bl.glcP-F
tagagtcgacctgcaatgaaaaaaatatttctattc

SEQ ID NO: 39 - DNA, 36 bp, Artificial Sequence
  Synthetic Bl.glcP-R
ccaagcttgcatgccctacatatccaaatgactctg

SEQ ID NO: 40 - DNA, 36 bp, Artificial Sequence
  Synthetic Ms.glcP-F
tagagtcgacctgcaatgaacgtgatcggcatcacc

SEQ ID NO: 41 - DNA, 36 bp, Artificial Sequence
  Synthetic Ms.glcP-R
ccaagcttgcatgcctcaatgccccagcgcttcggc

SEQ ID NO: 42 - DNA, 36 bp, Artificial Sequence
  Synthetic Zm.glf-F
tagagtcgacctgcaatgagttctgaaagtagtcag

SEQ ID NO: 43 - DNA, 36 bp, Artificial Sequence
  Synthetic Zm.glf-R
ccaagcttgcatgccctacttctgggagcgccacat

SEQ ID NO: 44 - DNA, 36 bp, Artificial Sequence
  Synthetic Vp.SglS-F
tagagtcgacctgcaatgtcgaacatcgagcacggc

SEQ ID NO: 45 - DNA, 36 bp, Artificial Sequence
  Synthetic Vp.SglS-R
ccaagcttgcatgcctcaccagaacagggtatacag

SEQ ID NO: 46 - DNA, 36 bp, Artificial Sequence
  Synthetic Kx.galP1-F
tagagtcgacctgcagtgaatgacgatactgtaaag

SEQ ID NO: 47 - DNA, 36 bp, Artificial Sequence
  Synthetic Kx.galP1-R
ccaagcttgcatgccttagcctcccggttgcccgat

SEQ ID NO: 48 - DNA, 36 bp, Artificial Sequence
  Synthetic Kx.galP2-F
tagagtcgacctgcagtgtcacagcccgtatcttcg

SEQ ID NO: 49 - DNA, 36 bp, Artificial Sequence
  Synthetic Kx.galP2-R
ccaagcttgcatgccctagcggccaatgcggcgcag

SEQ ID NO: 50 - DNA, 36 bp, Artificial Sequence
  Synthetic Kx.galP3-F
tagagtcgacctgcaatgcccgaagacgatctggtt

SEQ ID NO: 51 - DNA, 36 bp, Artificial Sequence
  Synthetic Kx.galP3-R
ccaagcttgcatgcctcaggcagatggacgtgtgga

SEQ ID NO: 52 - DNA, 36 bp, Artificial Sequence
  Synthetic Kx.galP4-F
tagagtcgacctgcaatggaaaatcagcccgcgccc

SEQ ID NO: 53 - DNA, 36 bp, Artificial Sequence
  Synthetic Kx.galP4-R
ccaagcttgcatgccttatttctcctgcggcagcgg

SEQ ID NO: 54 - DNA, 36 bp, Artificial Sequence
  Synthetic Kx.galP5-F
tagagtcgacctgcaatgcacaggcagcccggtatc

SEQ ID NO: 55 - DNA, 36 bp, Artificial Sequence
  Synthetic Kx.galP5-R
ccaagcttgcatgccctagtgctgttcgggcggcgc

SEQ ID NO: 56 - DNA, 36 bp, Artificial Sequence
  Synthetic Kx.gluP-F
tagagtcgacctgcaatgggcggtggtgtcctgcct

SEQ ID NO: 57 - DNA, 36 bp, Artificial Sequence
  Synthetic Kx.gluP-R
ccaagcttgcatgcctcacgcggcacgacccaaccc